(19) United States
(12) Patent Application Publication
(10) Pub. No.: US 2023/0363648 A1
(43) Pub. Date: Nov. 16, 2023

(54) DIAGNOSIS AND TREATMENT OF CANCERS APPLYING NEAR-INFRARED SPECTROSCOPY (NIRS)

(71) Applicant: Comron MALEKI, Thousand Oaks, CA (US)

(72) Inventor: Comron MALEKI, Thousand Oaks, CA (US)

(21) Appl. No.: 18/308,918

(22) Filed: Apr. 28, 2023

Related U.S. Application Data
(63) Continuation of application No. PCT/US21/56977, filed on Oct. 28, 2021.
(60) Provisional application No. 63/106,986, filed on Oct. 29, 2020.

(51) Int. Cl. A61B 5/00; A61B 18/20

(52) U.S. Cl. CPC A61B 5/0075; A61B 18/20; A61B 5/444; A61B 2018/00577

(57) ABSTRACT

Optical and/or thermal energy is directed onto target tissue, generating smoke. The smoke generated is collected and a spectrum of smoke generated from the tissue is measured. Based on the spectrum, the target tissue is characterized to be normal or non-normal such as cancerous.

### CROSS-REFERENCE

This application is a continuation of PCT Application No. PCT/US21/56977, filed Oct. 28, 2021; which claims the benefit of U.S. Provisional Application No. 63/106,986, filed Oct. 29, 2020; which applications are incorporated herein by reference in their entirety.

### BACKGROUND

The present disclosure relates to medical systems, devices, and methods, particularly for characterizing tissue during treatment procedures such as tissue ablation of cancerous tissue.

Diagnoses of cancerous and noncancerous tissues are typically confirmed by histological review of tissue samples. Samples are transported to laboratory in preserved form and processed for proper identification by specifically trained physicians. Diagnosed results are reported back to treating physician who will coordinate treatment of the tumor. In case of surgical options, the patient goes back to operating room, and the tumor will be removed and sent back to laboratory to be checked for complete removal. This process is time consuming, involves multiple health care professionals, and is costly. Hence, there are needs for improved ways to characterize tissues during tumor and cancerous tissue treatment.

The following references may be relevant: US 2016/002816 A1, WO 2018/115415 A1, U.S. Pat. No. 9,709,529 B2, U.S. Pat. No. 10,643,832 B2, U.S. Pat. No. 10,410,846 B2, and EP 3558149A1.

### SUMMARY

According to the present disclosure, heat may be focused on tissue to create smoke, the smoke gets sampled and analyzed, such as with near-infrared spectroscopy (NIRS) or other spectroscopy, and the complex different wavelengths for the smoke noticed, with tumor specific wavelength(s) being recognized. As long as these tumor specific wavelength(s) are present, focused heat and ablation will continue, leading to complete tumor removal. This principle can be applied to all tumors as long as their specific wavelength is identified.

Basal cell carcinoma and squamous cell carcinoma are two most common form of cancer of skin. Through NIRS or other spectroscopy, their specific pattern of wavelength(s) can be recognized, which can lead to diagnosis, as well as precise and complete removal of cancer with preservation of normal adjacent tissue. This technique can save time, may be more accurate, and can be far more economical than present routines and techniques. This technique may be applicable to other forms of growth (benign and cancerous) as well.

Aspects of the present disclosure provide methods of characterizing and treating tissue. In an exemplary method, a spectrum of smoke generated from tissue to be characterized may be measured, and the tissue may be characterized based on the measured spectrum of the smoke.

In some embodiments, the tissue to be characterized is heated to generate the smoke. The smoke may be generated by applying one or more of light or heat to the tissue. The one or more of the light or heat may be applied by an ablation device.

In some embodiments, the method further comprises ablating the tissue. Ablating the tissue may generate the smoke. The tissue may be ablated in situ. The method may further comprise excising the tissue from a patient. The ablated issue may comprise the excised tissue.

In some embodiments, the method further comprises capturing the smoke generated. The smoke may be captured in a cuvette. The spectrum of smoke may be measured at the cuvette.

In some embodiments, the tissue is characterized as cancerous or non-cancerous. The method may further comprise ablating the tissue and continuing ablating the tissue until the tissue is characterized as non-cancerous.

In some embodiments, the tissue to be characterized comprises skin. The tissue may be characterized as comprising basal cell carcinoma (BCC) cells, squamous cell carcinoma (SCC) cells, or normal cells.

In some embodiments, the measured spectrum is a near-infrared spectrum. The measured spectrum may be within a wavelength range of 1,300 nm to 1,600 nm. The measured spectrum may comprise a wavelength below 1,300 nm.

Further aspects of the present disclosure provide devices for charactering and treating tissue. An exemplary device may comprise an energy source to heat tissue and generate smoke therefrom and a spectrometer to measure a spectrum of the smoke generated from the tissue.

In some embodiments, the device further comprises a cuvette to capture the smoke generated from the tissue. The device may further comprise a negative pressure source to direct the smoke generated from the tissue to the cuvette.

In some embodiments, the energy source is configured to ablate tissue. The spectrometer may comprise a near-infrared spectrometer. The energy source may comprise one or more of a tissue cauterizer or laser.

In some embodiments, the device further comprises a processor coupled to the spectrometer. The processor may be configured to characterize the tissue based on the measured spectrum of the smoke. The processor may be configured to characterize the tissue as cancerous or non-cancerous. The tissue may comprise skin. The processor may be configured to characterize the tissue as comprising basal cell carcinoma (BCC) cells, squamous cell carcinoma (SCC) cells, or normal cells.

In some embodiments, the measured spectrum is a near-infrared spectrum. The measured spectrum may be within a wavelength range of 1,300 nm to 1,600 nm. The measured spectrum may comprise a wavelength below 1,300 nm.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the present disclosure are set forth with particularity in the appended claims. A better understanding of the features and advantages of embodiments of the present disclosure will be obtained by reference to the following detailed description that sets forth illustrative embodiments, in which the principles of the present disclosure are utilized, and the accompanying drawings of which:

FIG. 1 is a schematic diagram of a tissue characterization and/or treatment system according to embodiments of the present disclosure.

FIG. 2 is a flow chart of a tissue characterization and/or treatment method according to embodiments of the present disclosure;

FIG. 3 shows an image of an exemplary thermal source usable with the tissue characterization and/or treatment systems and methods according to embodiments of the present disclosure;

FIG. 4 shows an image of an exemplary vacuum source usable with the tissue characterization and/or treatment systems and methods according to embodiments of the present disclosure;

FIG. 5 shows an image of an exemplary smoke collection element usable with the tissue characterization and/or treatment systems and methods according to embodiments of the present disclosure;

FIG. 6 shows an image of an exemplary smoke collection element holder usable with the tissue characterization and/or treatment systems and methods according to embodiments of the present disclosure;

FIG. 7 shows an image of an exemplary light source usable with the tissue characterization and/or treatment systems and methods according to embodiments of the present disclosure;

FIG. 8 shows an image of an exemplary light conduction element usable with the tissue characterization and/or treatment systems and methods according to embodiments of the present disclosure;

FIG. 9 shows an image of an exemplary optical or spectral sensor usable with the tissue characterization and/or treatment systems and methods according to embodiments of the present disclosure;

FIG. 10 shows an image of an exemplary light conduction element usable with the tissue characterization and/or treatment systems and methods according to embodiments of the present disclosure;

FIG. 11 shows an image of an exemplary graphical user interface (GUI) usable with the tissue characterization and/or treatment systems and methods according to embodiments of the present disclosure;

FIG. 12 shows an image of a prototype tissue characterization and/or treatment systems and methods according to embodiments of the present disclosure;

FIG. 13 shows an image of an exemplary graphical user interface (GUI) usable with the tissue characterization and/or treatment systems and methods according to embodiments of the present disclosure;

FIGS. 14, 15, and 16 show spectral data graphs for BCC cancer cells, SCC cancer cells, and normal cells, respectively, according to embodiments of the present disclosure; and

FIG. 17 shows a schematic of an exemplary computer processing system for use with the tissue characterization and/or treatment systems and methods according to embodiments of the present disclosure.

### DETAILED DESCRIPTION

While various embodiments of the present disclosure have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the present disclosure described herein may be employed.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

Certain inventive embodiments herein contemplate numerical ranges. When ranges are present, the ranges include the range endpoints. Additionally, every sub range and value within the range is present as if explicitly written out. The term “about” or “approximately” may mean within an acceptable error range for the particular value, which will depend in part on how the value is measured or determined, e.g., the limitations of the measurement system. For example, “about” may mean within 1 or more than 1 standard deviation, per the practice in the art. Alternatively, “about” may mean a range of up to 20%, up to 10%, up to 5%, or up to 1% of a given value. Where particular values are described in the application and claims, unless otherwise stated the term “about” meaning within an acceptable error range for the particular value may be assumed.

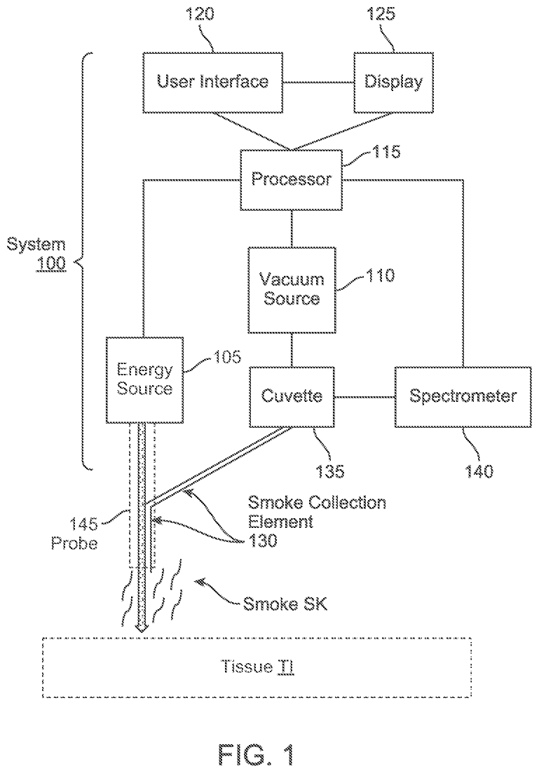

FIG. 1 shows an exemplary tissue characterization and/or treatment system 100. The system 100 may comprise an energy source 105 to direct energy toward the target tissue TI to generate smoke SK, a vacuum source 110 to apply negative pressure toward the smoke SK and collect the smoke SK at cuvette 135, a spectrometer 140 to measure the smoke SK collected at the cuvette 135. The energy source 105 may be an optical, thermal, and/or electrical energy source, for example, a laser or tissue cauterizer to generate focused heat.

The system 100 may further comprise a smoke collection element 130 coupled to the cuvette 135 to direct the negative pressure generated by the vacuum source 110 toward the smoke SK. The system 100 may further comprise a holder or holding element for the cuvette 135 which will typically be replaceable. The system 100 may further comprise a probe 145, such as a handheld probe or wand (for example, with a distal suction port and heat and/or light outlet, as well as control buttons and/or switches to operate the probe), which may integrate the smoke collection element 130 and/or energy conveying elements, such as fiber optics or thermal and/or electrical conductors, which may direct the energy directed from the energy source 105 to the tissue TI. The probe 145 may be a single use probe and may be removably coupled to the energy source 105 and/or smoke collection element 130 so that the probe 145 may be replaced after one or more uses. The other components of the system 100 outside of the probe 145 may be housed in system box or compartment.

The system 100 may further comprise a controller or processor 115 operatively coupled to the energy source 105, the vacuum source 110, and the spectrometer 140 to control such elements. The system 100 may further comprise a user interface 120 and a display 125 coupled to the processor 115 and/or user interface 120. The user interface 120 may be used by the operator to control the system 100, such as to operate the energy source 105 to direct energy onto the target tissue TI to generate the smoke SK. The user interface 120 may be used by the operator to generate a measurement of the smoke SK with the spectrometer 140 so as to characterize the target tissue TI. The spectrometer 140 may comprise a near-infrared spectrometer to measure a spectrum of the smoke SK within 1,300 nm to 1,600 nm, for example, though the spectrometer 140 may measure, alternatively or in combination, other wavelength ranges as well, including below 1,300 nm and/or any range within and including the entire infrared spectrum (e.g., 700 nm to 1 mm). Results of such measurement and tissue characterization may be shown by display 125. For instance, based on the measured spectrum, the target tissue TI the smoke SK is generated from may be categorized as normal or cancerous. In a further example, the cancerous tissue may be characterized by type, such as comprising basal cell carcinoma (BCC) cells and squamous cell carcinoma (SCC) cells where the target tissue TI is skin tissue. Smoke generated in each cell type category may have their own characteristic spectrum.

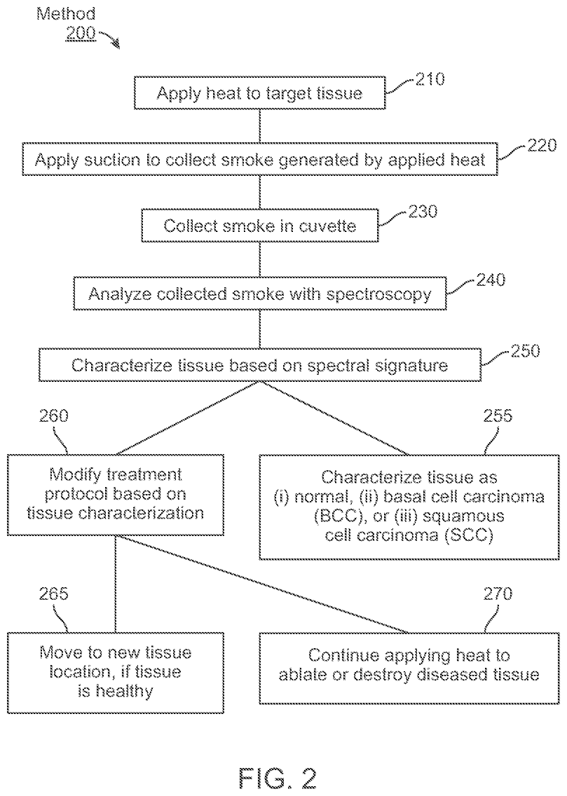

FIG. 2 is a flow chart of an exemplary tissue characterization and/or treatment method 200. In a step 210, heat may be applied to the target tissue, such as with the energy source 105 via the probe 145 to the target tissue TI, as operated by the user interface 120. In a step 220, suction may be applied to collect smoke generated by the applied heat, such as by the vacuum source 110. In a step 230, smoke is collected in a cuvette, such as the cuvette 135. In a step 240, the collected smoke is analyzed with spectroscopy, such as with the spectrometer 140. In a step 250, the target tissue is characterized based on the measured spectral signature of the smoke, such as with a processor 115. In a step 255, the target tissue is characterized as comprising normal cells, basal cell carcinoma (BCC) cells, or squamous cell carcinoma (SCC) cells, for example. In many embodiments, the tissue characterization results are provided to the operator, such as by being shown with the display 125.

In many embodiments, both tissue characterization and treatment are conducted contemporaneously, such as with the probe 145, and the heat applied can both generate the smoke that is analyzed and ablate or destroy target tissue, if applicable. In a step 260, the treatment protocol may be modified based on the tissue characterization. For example, focused heat may be applied to target tissue to first analyze the tissue and if the tissue is identified as comprising normal cells, the operator can move on to another tissue location, in a step 265, and if the tissue is identified as diseased, the operator can continue with applying focused heat using the same device to ablate or destroy the tissue, in a step 270. In many embodiments, the tissue characterization is provided by the system in real-time or near real-time (for example, in less than a minute, for instance, between 1-60 seconds, 1-30 seconds, 1-15 seconds, 1-10 seconds, 1-5 seconds, 1-4 seconds, 1-3 seconds, 1-2 seconds, around 1 second, or even less than 1 second) so that the overall tissue treatment procedure can be conducted in an expedited manner.

Although the above steps show method 200 in accordance with embodiments, a person of ordinary skill in the art will recognize many variations based on the teaching described herein. The steps may be completed in a different order. Steps may be added or omitted. Some of the steps may comprise sub-steps. Many of the steps may be repeated as often as beneficial.

One or more of the steps of method 200 may be performed with circuitry as described herein, for example, one or more of the processor or logic circuitry such as programmable array logic for a field programmable gate array. The circuitry may be programmed to provide one or more of the steps of the method 200, and the program may comprise program instructions stored on a computer readable memory or programmed steps of the logic circuitry such as the programmable array logic or the field programmable gate array, for example.

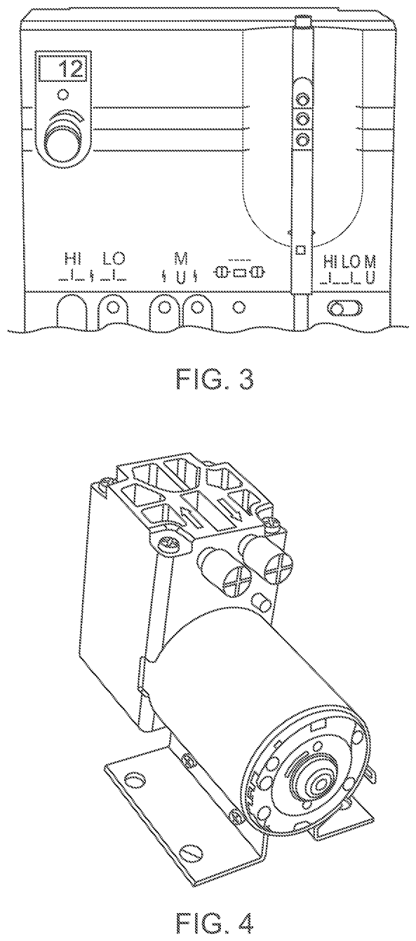

Referring back to the energy source 105, FIG. 3 shows an image of an exemplary thermal source usable with the tissue characterization and/or treatment system 100 and method 200 according to embodiments of the present disclosure. The exemplary thermal source shown by FIG. 3 is a CONMED Hyfrecator 2000 electrosurgical unit at power of “12” on a monopolar setting with fine tip cautery, available from CONMED Corporation of Largo, FL, and has been used by the inventor in a tested prototype. Other thermal sources may also be used alternatively or in combination.

Referring back to the vacuum source 110, FIG. 4 shows an image of an exemplary vacuum source usable with the tissue characterization and/or treatment system 100 and method 200. The exemplary vacuum source shown by FIG. 4 is a mini high pressure vacuum pump (12V-6 W) with a foot peddle control and has been used by the inventor in the tested prototype. Other vacuum sources may also be used alternatively or in combination.

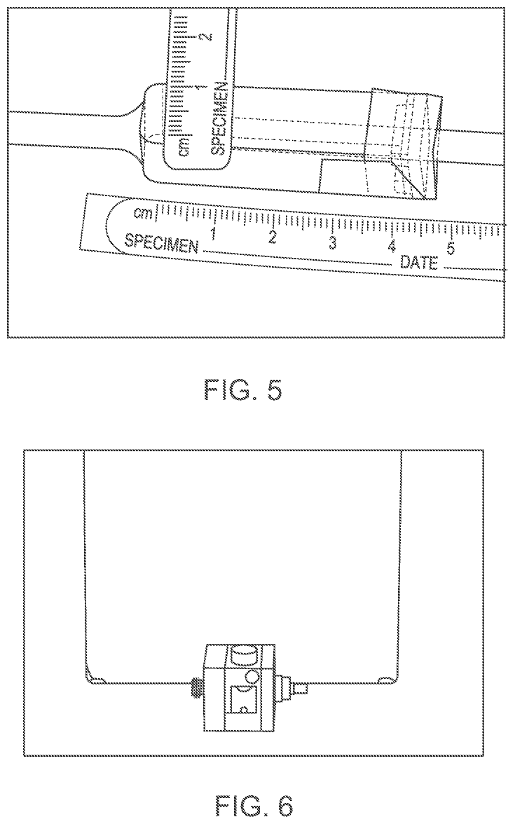

Referring back to the cuvette 135, FIG. 5 shows an image of an exemplary smoke collection element usable with the tissue characterization and/or treatment system 100 and method 200 and FIG. 6 shows an image of an exemplary smoke collection element holder usable with the tissue characterization and/or treatment system 100 and method 200. The exemplary smoke collection element shown by FIG. 5 is a cuvette with optical glass, such as a cuvette with the specifications 10×10×45 mm (CV19Q 3500) available from Thor Labs of Newton, New Jersey, and has been used by the inventor in the tested prototype. The cuvette may be made of glass, quartz, sapphire, zirconium, other optically transparent materials, and combinations thereof. The exemplary smoke collection element holder shown by FIG. 6 is a Direct-Attach Cuvette Holder (CUV-DA-HAL-Mini) available from Avantes USA of Louisville, CO, and has been used by the inventor in the tested prototype. Other cuvettes and/or smoke collection elements may also be used alternatively or in combination.

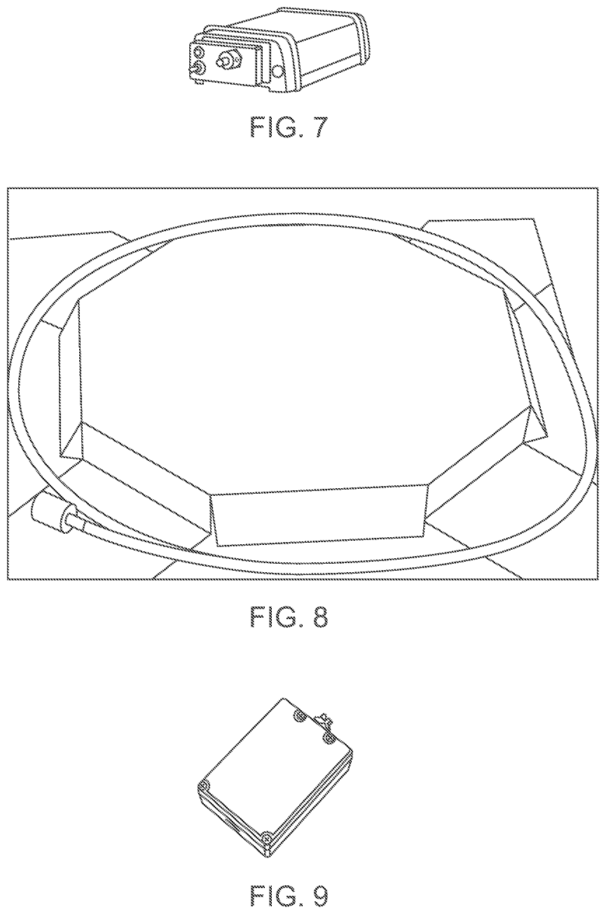

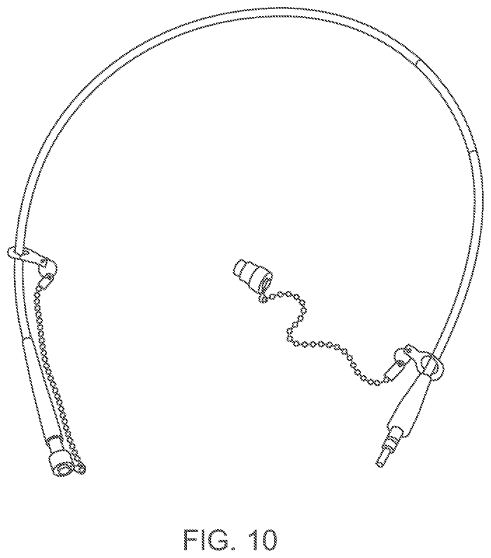

Referring back to the spectrometer 140, FIG. 7 shows an image of an exemplary light source usable with the spectrometer of the tissue characterization and/or treatment system 100 and method 200, FIG. 8 shows an image of an exemplary light conduction element usable with the tissue characterization and/or treatment system 100 and method 200, FIG. 9 shows an image of an exemplary optical or spectral sensor usable with the tissue characterization and/or treatment system 100 and method 200, and FIG. 10 shows an image of an exemplary light conduction element usable with the tissue characterization and/or treatment system 200 and method 100.

The exemplary light source shown by FIG. 7 is a tungsten light source, AvaLight-Hal-S-Mini (s/n: LS-1811005) with a wavelength range of 360 nm to 2,500 nm, available from Avantes USA of Louisville, CO, and has been used by the inventor in the tested prototype. Other compact, stabilized light sources may be used, alternatively or in combination. The light source was implemented in conjunction with the exemplary light conduction element or fiber optic shown by FIG. 8, which is an Avantes fiber optic FC-UVRI 400-1-ME (1609122) available from Avantes USA of Louisville, CO, and has been used by the inventor in the tested prototype. Other optical fibers or light conduction elements may also be used alternatively or in combination.

The exemplary optical or spectral sensor shown by FIG. 9 is an Si-Ware NeoSpectra-Module (PN: SWS62221.2.5 spectral range; Rev: 2.5; SN: K116241359) available from Si-Ware Systems, Inc. of Menlo Park, CA and has been used by the inventor in the tested prototype. This optical or spectral sensor was implemented in conjunction with the exemplary light conduction element or fiber optic shown by FIG. 10, which is an Si-Ware multimode optical fiber (Q MMJ-35-IRVIS400/440-3-0.5; SN: T21574431-09) available from Si-Ware Systems, Inc. of Menlo Park, CA and has been used by the inventor in the tested prototype. Other optical or spectral sensors, for anywhere in the infrared range, for example, may also be used alternatively or in combination.

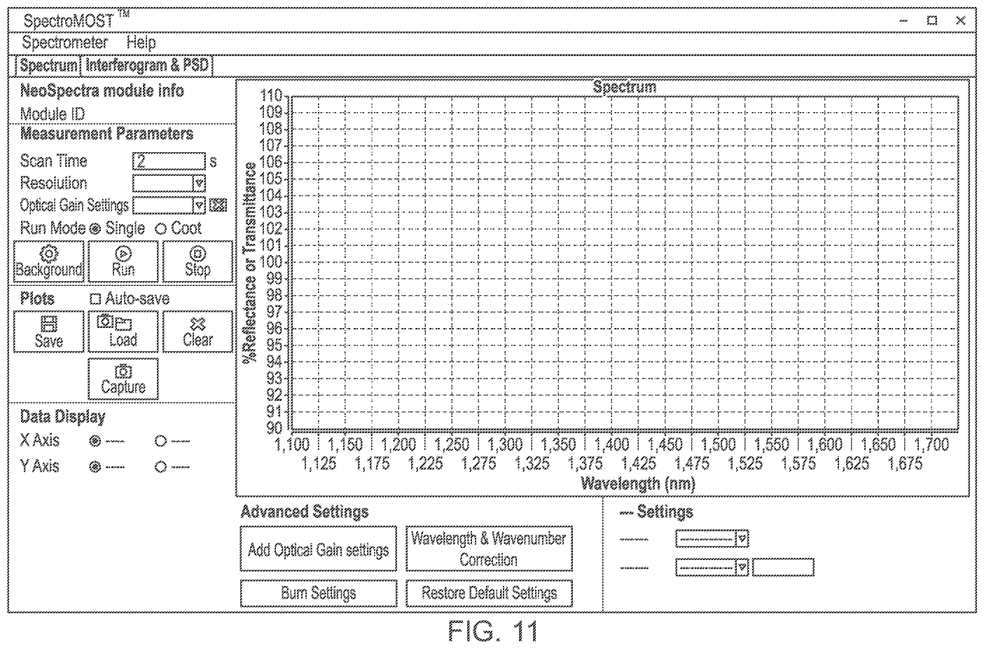

Referring now to the computer systems usable with the tissue characterization and/or treatment systems and methods of the present disclosure, a Dell laptop, available from Dell Inc. of Round Rock, Texas, was used by the inventor in the tested protype and used to implement the program to capture, display, and save spectral data. The software to capture, display, and save spectral data in the tested prototype was the NeoSpectra SpectroMOST software tool from available from Si-Ware Systems, Inc. of Menlo Park, CA. FIG. 11 shows an image of the graphical user interface (GUI) of this software tool. Other software package(s) may also be used alternatively or in combination.

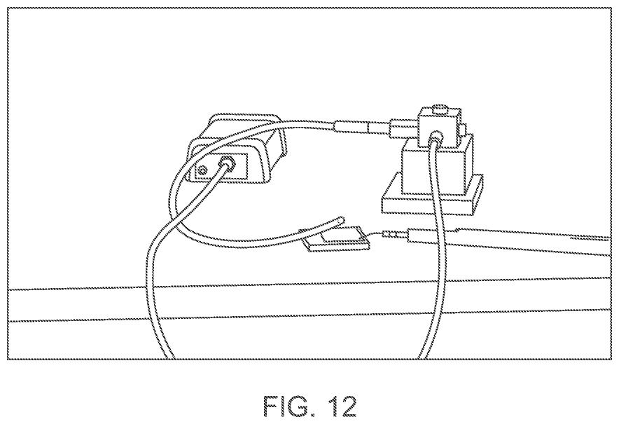

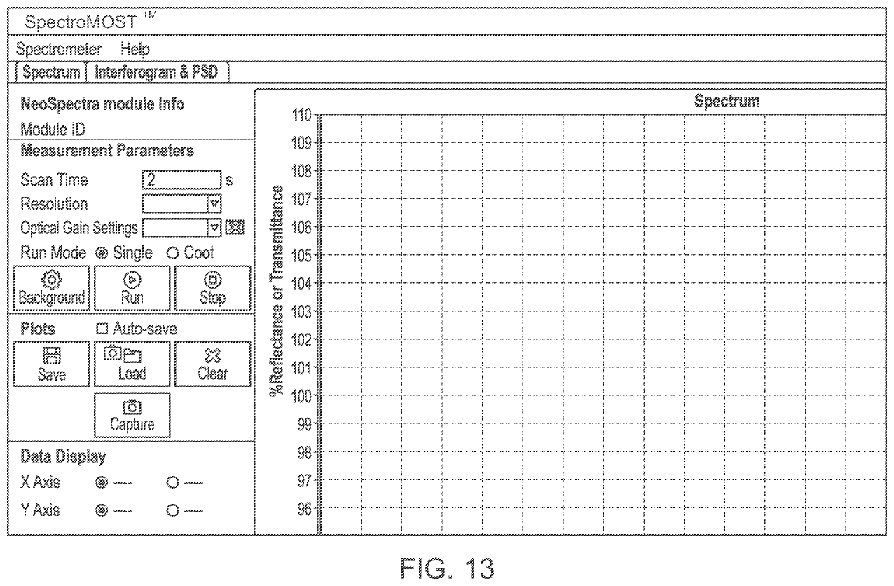

FIG. 12 shows an image of the tested prototype tissue characterization and/or treatment system. After the prototype system had been set up and devices turned on, the inventor allowed the Avantes light device, NeoSpectra sensors, and the computer a warm up period of about 25 minutes. The inventors also shielded the Avantes light source and cuvette from stray light sources in order to minimize external noise and to ensure high accuracy during the course of spectral measurements. The main steps in a measurement cycle included: (1) emptying the cuvette from residual smoke, (2) running the background test using SpectroMOST program, (3) after heat is applied to the tissue under test, clicking the Run button (of SpectroMOST) to capture spectral data and saving data, and (4) then clearing the screen of SpectrMOST to get ready for next data capture. Furthermore, after completion of each run and data capture, the smoke in the tubing (and cuvette) was removed by the vacuum source/pump, before starting the next run cycle. FIG. 13 shows an image of the SpectroMOST GUI, which illustrates the Background, Run and Clear buttons used in each measurement cycle.

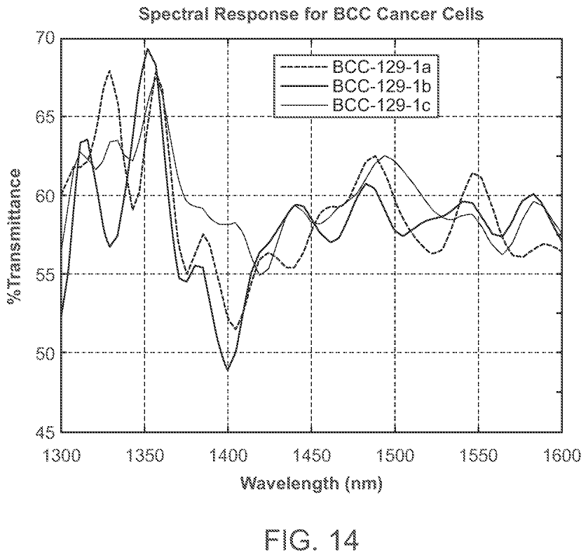

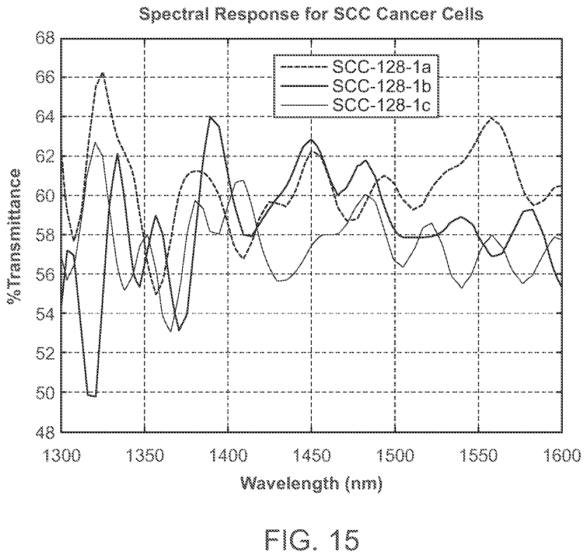

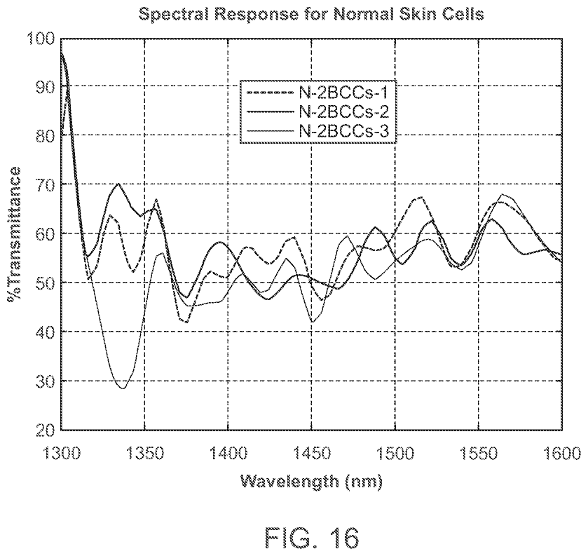

FIGS. 14, 15, and 16 show spectral data graphs (e.g., relative amplitude versus wavelength) for BCC cancer cells, SCC cancer cells, and normal cells, respectively. FIG. 14 shows a graph of transmission percentage versus wavelength for three basal cell carcinoma (BCC) samples (BCC-129-1a, BCC-129-1b, BCC-129-1c) within a wavelength range of 1,300 nm to 1,600 nm. FIG. 14 shows a graph of transmission percentage versus wavelength for three basal cell carcinoma (BCC) samples (BCC-129-1a, BCC-129-1b, BCC-129-1c) within a wavelength range of 1,300 nm to 1,600 nm. FIG. 15 shows a graph of transmission percentage versus wavelength for three squamous cell carcinoma (SCC) samples (SCC-128-1a, SCC-128-1b, SCC-128-1c) within a wavelength range of 1,300 nm to 1,600 nm. FIG. 16 shows a graph of transmission percentage versus wavelength for three normal skin cell samples (N-2BCCs-1, N-2BCCs-2, N-2BCCs-3) within a wavelength range of 1,300 nm to 1,600 nm.

As shown by FIGS. 14, 15, and 16, the spectral data for BCC, SCC, and normal cells may have unique characteristics usable to identify cell type in target tissue, for instance, as shown in the Table 1 below.

| Sample/ | BCC | SCC | Normal |
|---|---|---|---|
| Characteristic | (FIG. 14) | (FIG. 15) | (FIG. 16) |
| Greatest peak | 1,330 − 1,360 nm | 1,320 − 1,340 nm | 1,330 − 1,360 |
| | | | nm |
| Greatest though | 1,390 − 1,430 nm | 1,360 − 1,380 nm | 1,330 − 1,380 |
| | | | nm |
| Second greatest peak | 1,470 − 1,500 nm | 1,390 − 1,560 nm | 1,510 − 1,570 |
| | | | nm |
| Greatest peak | ~12-20 | ~10-15 | ~25 |
| to trough | | | |
| (percentage points) | | | |
| Range of | 67-69% | 63-66% | 69-70% |
| greatest peak | | | |
| Ranges of | 49-55% | 49-55% | 29-49% |
| greatest trough | | | |

As shown above in Table 1, different cell types may have different unique spectral characteristics which may be used to distinguish cancerous cells from normal cells and then to distinguish the type of cancerous cell. See, for example, the characteristics in italics in Table 1. For instance, the greatest troughs for normal cells are usually much lower (29-49%) than that for cancerous cells (49-55%) and the range from highest peak to lowest trough is greater (about 25 percentage points) than that for cancerous cells (about 12-20 percentage points for BCC cells and about 10-15 percentage points for SCC cells), and SCC cells appear to have their greatest peaks and troughs generally shifted to lower wavelength ranges versus BCC cells, as well as having lower greatest peaks (63-66% for SCC cells versus 67-69% for BCC cells).

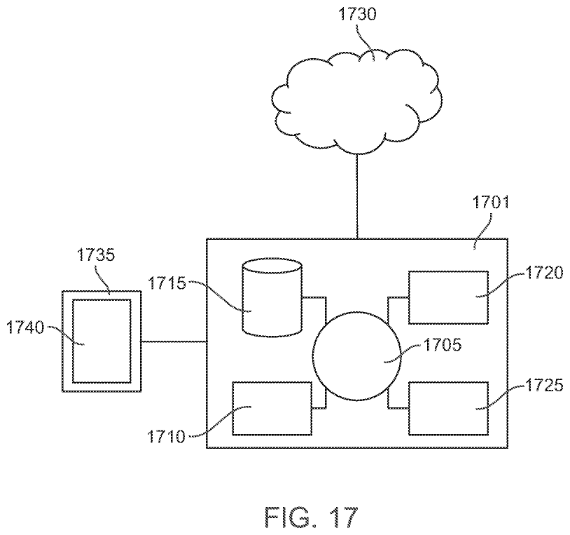

The present disclosure also provides computer systems that are programmed to implement methods of the present disclosure. FIG. 17 shows a computer system 1701 that is programmed or otherwise configured to implement the methods of the present disclosure, including method 200 described above. The computer system 1701 can regulate various aspects of tissue characterization and/or treatment devices and systems of the present disclosure, such as, for example, one or more elements of system 100. The computer system 1701 can be an electronic device of a user or a computer system that is remotely located with respect to the electronic device. The electronic device can be a mobile electronic device.

The computer system 1701 may include a central processing unit (CPU, also “processor” and “computer processor” herein) 1705, which can be a single core or multi core processor, or a plurality of processors for parallel processing. The computer system 1701 may also include memory or memory location 1710 (e.g., random-access memory, read-only memory, flash memory), electronic storage unit 1715 (e.g., hard disk), communication interface 1720 (e.g., network adapter) for communicating with one or more other systems, and peripheral devices 1725, such as cache, other memory, data storage and/or electronic display adapters. The memory 1710, storage unit 1715, interface 1720, and peripheral devices 1725 may be in communication with the CPU 1705 through a communication bus (solid lines), such as a motherboard. The storage unit 1715 can be a data storage unit (or data repository) for storing data. The computer system 1701 can be operatively coupled to a computer network (“network”) 1730 with the aid of the communication interface 1720. The network 1730 can be the Internet, an internet and/or extranet, or an intranet and/or extranet that is in communication with the Internet. The network 1730 in some cases is a telecommunication and/or data network. The network 1730 can include one or more computer servers, which can enable distributed computing, such as cloud computing. The network 1730, in some cases with the aid of the computer system 1701, can implement a peer-to-peer network, which may enable devices coupled to the computer system 1701 to behave as a client or a server.

The CPU 1705 can execute a sequence of machine-readable instructions, which can be embodied in a program or software. The instructions may be stored in a memory location, such as the memory 1710. The instructions can be directed to the CPU 1705, which can subsequently program or otherwise configure the CPU 1705 to implement methods of the present disclosure. Examples of operations performed by the CPU 1705 can include fetch, decode, execute, and writeback.

The CPU 1705 can be part of a circuit, such as an integrated circuit. One or more other components of the system 1701 can be included in the circuit. In some cases, the circuit is an application specific integrated circuit (ASIC).

The storage unit 1715 can store files, such as drivers, libraries and saved programs. The storage unit 1715 can store user data, e.g., user preferences and user programs. The computer system 1701 in some cases can include one or more additional data storage units that are external to the computer system 1701, such as located on a remote server that is in communication with the computer system 1701 through an intranet or the Internet.

The computer system 1701 can communicate with one or more remote computer systems through the network 1730. For instance, the computer system 1701 can communicate with a remote computer system of a user (e.g., an operator or a surgeon). Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 1701 via the network 1730.

Methods as described herein can be implemented by way of machine (e.g., computer processor) executable code stored on an electronic storage location of the computer system 1701, such as, for example, on the memory 1710 or electronic storage unit 1715. The machine executable or machine readable code can be provided in the form of software. During use, the code can be executed by the processor 1705. In some cases, the code can be retrieved from the storage unit 1715 and stored on the memory 1710 for ready access by the processor 1705. In some situations, the electronic storage unit 1715 can be precluded, and machine-executable instructions are stored on memory 1710.

The code can be pre-compiled and configured for use with a machine having a processer adapted to execute the code or can be compiled during runtime. The code can be supplied in a programming language that can be selected to enable the code to execute in a pre-compiled or as-compiled fashion.

Aspects of the systems and methods provided herein, such as the computer system 1701, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 1701 can include or be in communication with an electronic display 1735, for example, the display 125, that comprises a user interface (UI) 1740, for example, the user interface 120, for providing, for example, control of the system 100 and characterization or analysis results for the targeted tissue. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 1705. The algorithm can, for example, a machine learning algorithm or a Bayesian optimization to classify spectral measurements into categories including healthy and non-healthy tissue, including sub-categories for the disease state or cell composition of the tissue. These machine learning algorithms and/or classifier models may be trained by input from one or more operators which can verify or update the tissue classification provided by the algorithm/model based on their independent assessment of one or more spectral patterns of the analyzed tissue, thereby providing improvements to the machine learning algorithms and/or classifier over time. The training sets for these machine learning algorithms and/or classifier models may comprise the spectral patterns of various tissue that have been analyzed and/or input from the operator or other human analysis of the spectral patterns during or after procedures, e.g., training via supervised learning. The machine learning algorithms and/or classifier models may also be trained via unsupervised learning from the same or different training data set.

While preferred embodiments of the present disclosure have been shown and described herein, it will be obvious to those skilled in the art that such embodiments are provided by way of example only. Numerous variations, changes, and substitutions will now occur to those skilled in the art without departing from the present disclosure. It should be understood that various alternatives to the embodiments of the present disclosure described herein may be employed in practicing the present disclosure. It is intended that the following claims define the scope of the invention and that methods and structures within the scope of these claims and their equivalents be covered thereby.

What is claimed is:

1. A method of characterizing and treating tissue, the method comprising:
measuring a spectrum of smoke generated from tissue to be characterized; and
characterizing the tissue based on the measured spectrum of the smoke.

2. The method of claim 1, further comprising heating the tissue to be characterized to generate the smoke.

3. The method of claim 2, wherein the smoke is generated by applying one or more of light or heat to the tissue.

4. The method of claim 3, wherein the one or more of the light or heat is applied by an ablation device.

5. The method of claim 1, further comprising ablating the tissue.

6. The method of claim 5, wherein ablating the tissue generates the smoke.

7. The method of claim 5, wherein the tissue is ablated in situ.

8. The method of claim 5, further comprising excising the tissue from a patient, and wherein the ablated issue comprises the excised tissue.

9. The method of claim 1, further comprising capturing the smoke generated.

10. The method of claim 9, wherein the smoke is captured in a cuvette.

11. The method of claim 10, wherein the spectrum of smoke is measured at the cuvette.

12. The method of claim 1, wherein the tissue is characterized as cancerous or non-cancerous.

13. The method of claim 12, further comprising ablating the tissue and continuing ablating the tissue until the tissue is characterized as non-cancerous.

14. The method of claim 1, wherein the tissue comprises skin.

15. The method of claim 14, wherein the tissue is characterized as comprising basal cell carcinoma (BCC) cells, squamous cell carcinoma (SCC) cells, or normal cells.

16. The method of claim 1, wherein the measured spectrum is a near-infrared spectrum.

17. The method of claim 16, wherein the measured spectrum is within a wavelength range of 1,300 nm to 1,600 nm.

18. The method of claim 16, wherein the measured spectrum comprises a wavelength below 1,300 nm.

19. A device for charactering and treating tissue, the device comprising:
an energy source to heat tissue and generate smoke therefrom;
a spectrometer to measure a spectrum of the smoke generated from the tissue.

20. The device of claim 19, further comprising a cuvette to capture the smoke generated from the tissue.

21. The device of claim 20, further comprising a negative pressure source to direct the smoke generated from the tissue to the cuvette.

22. The device of claim 19, wherein the energy source is configured to ablate tissue.

23. The device of claim 19, wherein the spectrometer comprises a near-infrared spectrometer.

24. The device of claim 19, wherein the energy source comprises one or more of a tissue cauterizer or laser.

25. The device of claim 19, further comprising a processor coupled to the spectrometer, the processor being configured to characterize the tissue based on the measured spectrum of the smoke.

26. The device of claim 25, wherein the processor is configured to characterize the tissue as cancerous or non-cancerous.

27. The device of claim 26, wherein the tissue comprises skin, and wherein the processor is configured to characterize the tissue as comprising basal cell carcinoma (BCC) cells, squamous cell carcinoma (SCC) cells, or normal cells.

28. The device of claim 19, wherein the measured spectrum is a near-infrared spectrum.

29. The device of claim 28, wherein the measured spectrum within a wavelength range of 1,300 nm to 1,600 nm.

30. The device of claim 28, wherein the measured spectrum comprises a wavelength below 1,300 nm.
